(19) United States
(12) Patent Application Publication
(10) Pub. No.: US 2005/0267466 A1
(43) Pub. Date: Dec. 1, 2005

(54) Thermocouple electrode

(76) Inventors: Douglas A. Staunton, Kalamazoo, MI (US); Eland Cramlet, Portage, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/138,192

(22) Filed: May 26, 2005

Related U.S. Application Data
(60) Provisional application No. 60/574,478, filed on May 26, 2004.

(51) Int. Cl. A61B 18/14

(52) U.S. Cl. 606/41

(57) ABSTRACT

The invention provides an electrode assembly for a system for performing an electrosurgical procedure using electrical energy. The electrode assembly includes an electrode defining a blind aperture for receiving a thermocouple and formed from nitinol. The nitinol electrode is super-elastic, resistant to corrosion, and bio-compatible. In the exemplary embodiment of the invention, a thermocouple is disposed in the blind aperture.

### CROSS REFERENCE TO RELATED APPLICATION

This application claims the benefit of U.S. Provisional Patent Application Ser. No. 60/574,478 for a RADIO REQUENCY GENERATOR, filed on May 26, 2004, which is hereby incorporated by reference in its entirety.

### BACKGROUND OF THE INVENTION

1. Field of the Invention

The invention relates to a system for performing an electrosurgical procedure using an electric stimulator integrated with a radiofrequency generator and more particularly to a thermocouple electrode for such a system.

2. Description of the Prior Art

In the field of electrosurgery, it is well known to contact an electrode to a target nerve tissue area of a patient for delivery of radiofrequency output through the electrode to the target nerve tissue area. The delivery of the radiofrequency output through the electrode to the target nerve tissue area is used to cut or coagulate the target nerve tissue area or to create a lesion in the target nerve tissue area. Generally the electrode is in communication with a control unit for controlling the delivery of the radiofrequency output to the electrode. More specifically, radiofrequency output is delivered to the target nerve tissue area to create a lesion to interrupt nerve communication. Lesion creation generally includes the steps of sensory stimulation, motor stimulation, and lesion creation. Sensory stimulation is used to facilitate the proper placement of the electrode before creating the lesion. Motor stimulation is used to avoid proximity of the electrode to the motor nerve before lesion creation to prevent inadvertent damage. And lesion creation exposes the target nerve tissue area to radiofrequency output to create the lesion to interrupt a nerve path. Alternatively, radiofrequency energy may be applied with a low duty cycle to prevent creation of a lesion, but still deliver an intense electric field to the target tissue. This intense electric field influences nerve fiber transmission and can provide a more conservative treatment option to lesion creation.

During the course of the procedure, it is necessary to alternate between electrical stimulation pulses and radiofrequency output. Each of the sensory stimulation, the motor stimulation, and the lesion creation utilize different electrical outputs. In addition, the stimulation and radiofrequency specifications vary with patients and procedures. Specific examples of such specifications which require changing, among others, include amplitude, frequency, temperature, duration, and radiofrequency and on time settings.

### SUMMARY OF THE INVENTION

The invention provides an electrode assembly for a system for performing an electrosurgical procedure using electrical energy. The electrode assembly includes an electrode defining a blind aperture for receiving a thermocouple and formed from nitinol. The nitinol electrode is super-elastic, resistant to corrosion, and bio-compatible. In the exemplary embodiment of the invention, a thermocouple is disposed in the blind aperture.

### BRIEF DESCRIPTION OF THE DRAWINGS

Advantages of the present invention will be readily appreciated, as the same becomes better understood by reference to the following detailed description when considered in connection with the accompanying drawings wherein:

FIG. 1 is a perspective view of an exemplary system for use in electrosurgical procedures;

FIG. 2 is a cross-sectional view of an electrode according to the exemplary embodiment of the invention;

FIG. 3 is a cross-sectional view of an electrode assembly according to the exemplary embodiment of the invention;

FIG. 4 is a detail view taken along detail line 4 in FIG. 3; and

FIG. 5 is an exploded view of the electrode assembly.

### DETAILED DESCRIPTION OF THE EXEMPLARY EMBODIMENT

A system 10 for generating radiofrequency output for use in electrosurgical procedures includes an electrode 12 for contacting a target nerve tissue area of a patient and for delivering the electrical energy to the target nerve tissue area. The electrical energy includes stimulation energy for performing stimulation to assure proper placement of the first electrode as well as radiofrequency energy for creation of a lesion.

The system 10 further includes a control unit 14 for controlling the delivery of the electrical energy to the electrode 12 and a screen unit 16 for displaying a plurality of screen views for providing operator inputs to the control unit 14 to control the delivery of electrical energy to the electrode 12. The screen unit 16 can include a touch sensitive screen for navigating through the plurality of screen views and for providing inputs to the control unit 14 for controlling the delivery of electrical energy to the electrode 12.

In the exemplary embodiment of the invention, a multi-function hand controller 18 is disposed in communication with the control unit 14 and remote from the screen unit 16 for providing inputs to the control unit 14. An operator may position the multi-function hand controller 18 at the patient's side and enter inputs to the control unit 14 by either of the multi-function hand controller 18 and the screen unit 16. In addition, the multi-function hand controller 18 corresponds to the screen unit 16 for entering inputs in parallel to the control unit 14. In other words the operator may be located at the patient's side and not in a line of sight with the screen unit 16 while providing inputs to the control unit 14 with the multi-function hand controller 18 to perform the electrosurgical procedure. Because the multi-function hand controller 18 operates in parallel with the screen unit 16, the operator may enter some inputs to the control unit 14 through the screen unit 16 and enter other inputs to the control unit 14 through the multi-function hand controller 18. The control unit 14 includes software and inputs to the control unit 14 through either of the screen unit 16 and the multi-function hand controller 18 controls the software, as will be discussed further below.

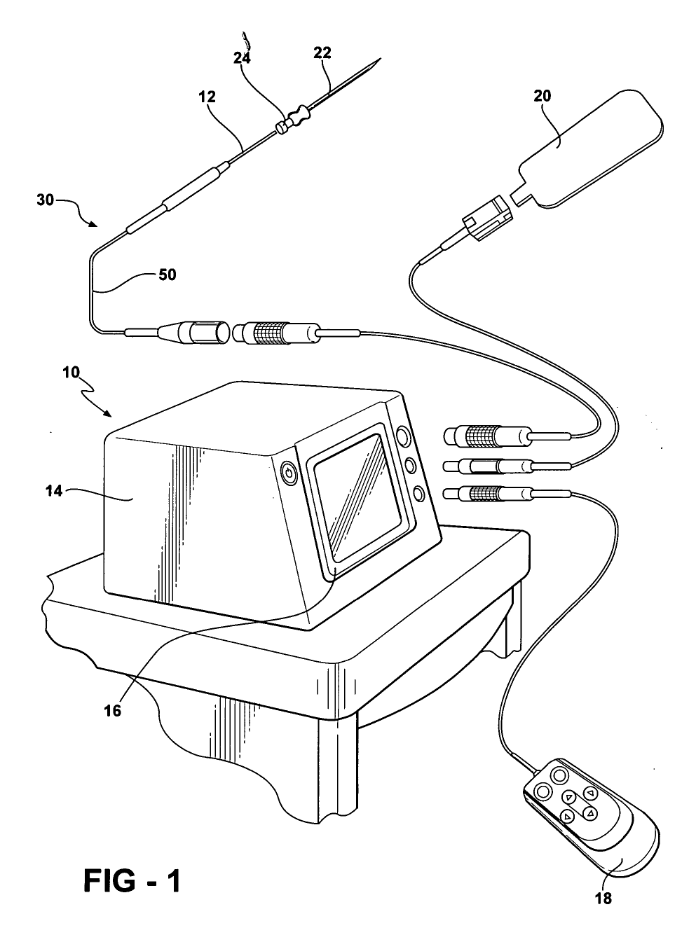

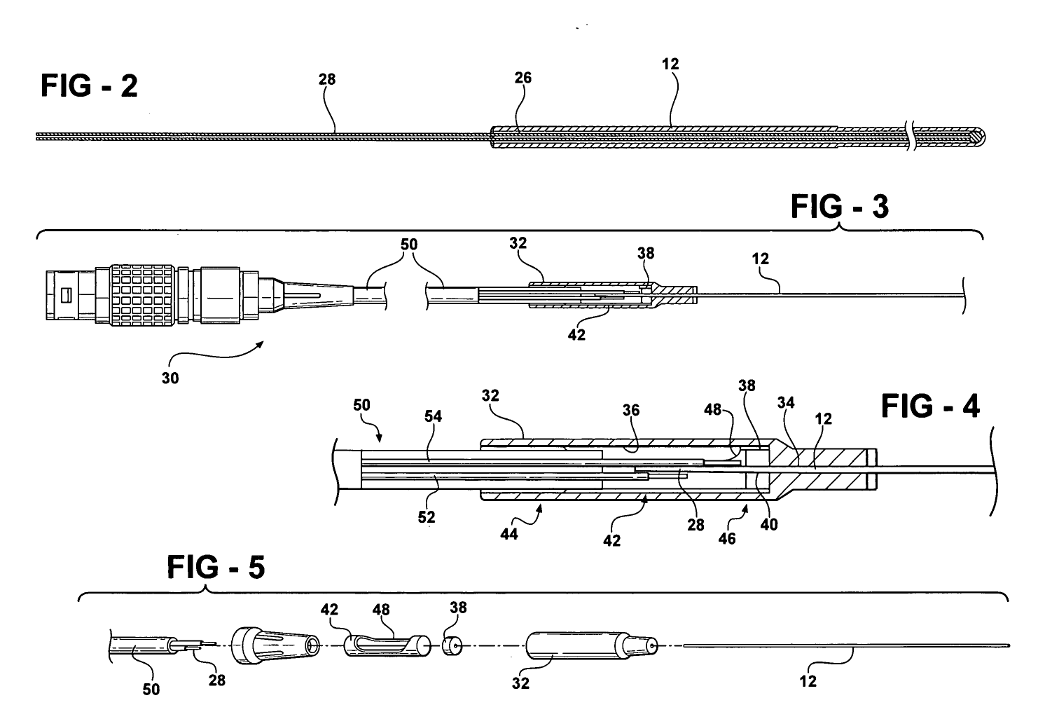

In operation, a second electrode 20 is placed in contact with the patient to complete the electrical circuit. In the embodiment shown in FIG. 1, the second electrode 20 is a pad for contacting the patient's skin. Alternatively, the second electrode 20 may be in the form of an electrode similar to the electrode 12. A radiofrequency generator is in communication with the electrode 12 and is controlled by the control unit 14 for providing the stimulation and radiofrequency output to the electrode 12. The second electrode 20 is in communication with the radiofrequency generator and thus completes the electrical circuit from the radiofrequency generator through the electrode 12 and through the patient, returning through the second electrode 20 to the radiofrequency generator. The electrode 12 and the second electrode 20 may be of the type well known in the art for performing monopolar or bipolar electrosurgery.

The system 10 also includes a cannula 22 for providing access for the electrode 12 to the target nerve tissue area. A stylet 24 is coaxially insertable into and removable from the cannula 22 for providing structural rigidity for insertion of the cannula 22 into the target nerve tissue area and for removal of the stylet 24 after insertion of the cannula 22 into the target nerve tissue area. The electrode 12 is in communication with the radiofrequency generator for insertion into the cannula 22 after removal of the stylet 24 to contact the target nerve tissue area for delivering the electrical energy to the target nerve tissue area. Other aspects of the exemplary system 10 are shown in a copending application for a SYSTEM AND METHOD FOR CONTROLLING ELECTRICAL STIMULATION AND RADIOFREQUENCY OUTPUT FOR USE IN AN ELECTROSURGICAL PROCEDURE, filed May 5, 2005, which is hereby incorporated by reference in its entirety.

The exemplary electrode 12 includes a blind aperture 26. A thermocouple 28 is received in the blind aperture 26. A tip of the thermocouple 28 should remain in contact with the closed end of the blind aperture 26 between 30° C. and 101° C. Blue stycast adhesive, or an equivalent, can be used to close the open end of the blind aperture 26 after the thermocouple 28 has been received. Preferrably, the outer surface of the electrode 12 is electro-polished.

The exemplary electrode 12 is formed from nitinol. It has been observed that, during use, the electrode 12 may be subjected to stress and plastic deformation. Nitinol enhances the capacity of the electrode 12 to sustain stress and plastic deformation. As a result, nitinol enhances the useful life of the electrode 12. Furthermore, nitinol is resistant to corrosion and is bio-compatible.

The electrode 12 is part of an exemplary electrode assembly 30. The electrode assembly 30 includes an outer housing 32 defining a first aperture 34 and a second aperture 36. The second aperture 36 is larger than and coaxial with the first aperture 34. The electrode 12 is received in both of the first and second apertures 34, 36 and extends out of the first aperture 34.

The electrode assembly 30 also includes an inner housing 42. The inner housing 42 includes an outside diameter corresponding to the second aperture 36 and is received in the second aperture 36. The inner housing 42 is tube-like and extends between first and second ends 44, 46. The inner housing 42 includes an aperture 48 extending transverse to the centered longitudinal axis of the inner housing 42.

The electrode assembly 30 also includes a guide ring 38. The guide ring 38 is disposed adjacent the first aperture 34 and receives the electrode 12 in an aperture 40. The guide ring 38 is positioned in the inner housing 42. The electrode assembly 30 also includes a cable assembly 50 having first and second wires 52, 54. The cable assembly 50 is received in the inner and outer housings 42, 32. The wire 52 is soldered to the thermocouple 28 and the wire 54 is soldered to the electrode 12. After soldering, epoxy and hardener can be dispensed in the inner housing 42.

The guide ring 38 and the inner housing 42 can be connected to one another with epoxy. Also, the inner and outer housings 42, 32 can be connected to one another with epoxy.

Obviously, many modifications and variations of the present invention are possible in light of the above teachings. The invention may be practiced otherwise than as specifically described within the scope of the appended claims. In addition, the reference numerals in the claims are merely for convenience and are not to be read in any way as limiting.

1. An electrode assembly for a system for performing an electrosurgical procedure using electrical energy comprising:
an electrode defining a blind aperture for receiving a thermocouple and formed from nitinol.

2. The electrode assembly of claim 1 wherein said electrode is further defined as being formed from super-elastic nitinol.

3. The electrode assembly of claim 2 wherein said electrode is further defined as being bio-compatible.

4. The electrode assembly of claim 3 further comprising:
a thermocouple received in said blind aperture.

5. The electrode assembly of claim 4 further comprising:
an outer housing defining a first aperture and a second aperture larger than and coaxial with said first aperture, wherein electrode being received in both of said first and second apertures and extending out of said first aperture.

6. The electrode assembly of claim 5 further comprising:
an inner housing received in said second aperture and extending tube-like and extends between first and second ends and having an aperture extending transverse to a centered longitudinal axis of the inner housing.

7. The electrode assembly of claim 6 further comprising:
a guide ring positioned in the inner housing adjacent said first aperture and receiving the electrode.

8. The electrode assembly of claim 7 further comprising:
a cable assembly having first and second wires and received in the inner and outer housings wherein said first wire is soldered to said thermocouple and said second wire is soldered to said electrode.
